(19) United States
(12) Patent Application Publication
(10) Pub. No.: US 2022/0151770 A1
(43) Pub. Date: May 19, 2022

(54) SUCTION-BASED TOOL FOR POSITIONING INTRAOCULAR IMPLANTS

(71) Applicant: THE REGENTS OF THE UNIVERSITY OF CALIFORNIA, Oakland, CA (US)

(72) Inventors: Xiangfeng DUAN, Los Angeles, CA (US); Yu HUANG, Los Angeles, CA (US); Chao MA, Los Angeles, CA (US)

(73) Assignee: THE REGENTS OF THE UNIVERSITY OF CALIFORNIA, Oakland, CA (US)

(21) Appl. No.: 17/528,080

(22) Filed: Nov. 16, 2021

Related U.S. Application Data
(60) Provisional application No. 63/114,338, filed on Nov. 16, 2020.

(51) Int. Cl. A61F 2/16; A61B 17/30

(52) U.S. Cl. CPC A61F 2/1664; A61B 2017/306; A61B 17/30

(57) ABSTRACT

In some embodiments, a suction-based tool for positioning an intraocular implant includes a tool body defining an internal channel; a suction extension extending through the internal channel; and a suction attachment connected to an end of the suction extension. In additional embodiments, a suction-based tool for positioning an intraocular implant includes: a tool body including a tool tip; a joint at an end of the tool tip; and a suction attachment connected to the tool tip via the joint.

### CROSS-REFERENCE TO RELATED APPLICATIONS

The present application claims priority to U.S. Provisional Patent Application No. 63/114,338 filed Nov. 16, 2020, the contents of which are incorporated herein by reference in their entirety.

### STATEMENT REGARDING FEDERALLY SPONSORED RESEARCH OR DEVELOPMENT

This invention was made with government support under Grant Number EY024065, awarded by the National Institutes of Health. The government has certain rights in the invention.

### TECHNICAL FIELD

This disclosure relates generally to a tool for positioning of implants during intraocular surgery.

### SUMMARY

In some embodiments, a suction-based tool for positioning an intraocular implant includes a tool body defining an internal channel; a suction extension extending through the internal channel; and a suction attachment connected to an end of the suction extension.

In additional embodiments, a suction-based tool for positioning an intraocular implant includes: a tool body including a tool tip; a joint at an end of the tool tip; and a suction attachment connected to the tool tip via the joint.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1(a) illustrates an example IOL injector used to insert an IOL into a capsular bag.

FIG. 1(b) illustrates an example of how an embodiment of a suction-based tool is inserted, secures to optics of an IOL, and accurately positions and aligns the IOL within the capsular bag.

FIG. 2 is an example illustration of automated IOL detection and alignment according to embodiments.

FIG. 3(a), FIG. 3(b) and FIG. 3(c) illustrate operational aspects of an example embodiment of a suction-based tool including a flexible extension of the tool with a compliant, suction-based end-effector.

FIG. 4 illustrates another example embodiment of a suction-based tool having a rigid tool body connected via a mechanically passive or actuated joint to a suction attachment.

### DETAILED DESCRIPTION

Embodiments of this disclosure are directed to a method and a tool to position an intraocular implant (e.g., an intraocular lens (IOL)) in a final stage of cataract surgery. In some embodiments, a non-marring, suction-based method and tool is provided to secure onto the IOL. By allowing both push and pull forces, IOL positioning is facilitated for robotic and human control but also provides a less intrusive manner to position the IOL. Because the suction-based tool adheres to an IOL optical center, there is no need to access haptics of the IOL and therefore the tool need not ever touch an iris, as is often the case in comparative positioning methods and tools. Future improved IOL designs may be promoted through use of the proposed tool concept. The present embodiments may be used in conjunction with one or more of other methods and apparatuses developed by the present Applicant, including U.S. patent application Ser. No. 16/487,074, U.S. patent application Ser. No. 16/982,506, U.S. patent application Ser. No. 17/021,925, U.S. patent application Ser. No. 17/049,909, U.S. patent application Ser. No. 17/052,758, U.S. Patent Application No. 63/143,336 filed Jan. 29, 2021 and U.S. Patent Application No. 63/210,256 filed Jun. 14, 2021, the contents of all such applications being incorporated herein by reference in their entirety.

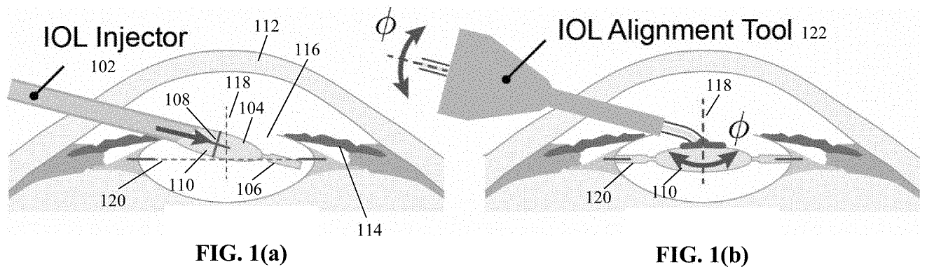

More generally with respect to the present embodiments, the final stage of cataract surgery involves inserting an IOL into a capsular bag and accurately positioning and aligning the IOL. Comparative tools used by a surgeon for IOL positioning and aligning include a pick or micro-forceps in addition to an automated injector used to insert the IOL implant into the capsular bag. However, these tools are specifically designed for other surgical procedures. These and other problems that have been recognized by the present applicant are illustrated in FIG. 1(a).

For example, a surgeon may inject an IOL into a capsular bag 116 behind an iris 114 and a cornea 112 of an eye with an IOL injector 102 and then continue to use the injector to push the IOL 110 into position as shown in FIG. 1(a). After injection, the surgeon may use a micro-forceps or another similar grasping tool to grab onto IOL haptics and position the IOL 110. More particularly, as shown in FIG. 1(a), an IOL 110 typically includes two components: optics 104 and haptics 106. The optics 104 are the functional component of the IOL 110 and should remain undamaged such that passage of light is unobstructed. The haptics 106 are “arms” or extensions on a side of the IOL 110, which are specifically designed to facilitate accurate positioning and aligning of the IOL 110 within the eye. As further shown in FIG. 1(a), the IOL 110 is generally vertically and horizontally centered around axes 108 (only two dimensions are shown for ease of illustration, but it should be apparent that the principles described here apply to three dimensions also).

After injecting the IOL 110, its axes 108 may need to be further positioned and aligned with a horizontal axis 120 corresponding to the general plane of iris 114, as well as centered with respect to the opening of the eye generally shown by axis 118. The use of tools—which are specifically designed for other surgical procedures—for positioning and aligning an IOL have the potential of damaging optics 104 of the IOL through tool touching or improper use of an injector or a grasping tool. Further, the use of such tools—which are specifically designed for human use—may be unsuitable for use by a robotic surgical system.

One example method and tool for IOL injection, positioning, and alignment according to embodiments is illustrated in FIG. 1(b). In this embodiment, after an IOL has been injected (for example using an injector 102 as shown in FIG. 1(a)), a suction-based tool 122 according to embodiments is inserted and accurately positions and aligns the IOL 110 within the capsular bag. More particularly, as shown in FIG. 1(b), after injection, the tool 122 attaches directly to optics 104 of IOL 110. Thereafter, by manipulation of tool 122, the IOL is able to be rotated by angles shown as 1 so as to align axes 108 of the IOL 110 with axes 118 and 120 of the eye. After being so positioned, the suction may be deactivated and the tool 122 removed, leaving the IOL 110 in an aligned position.

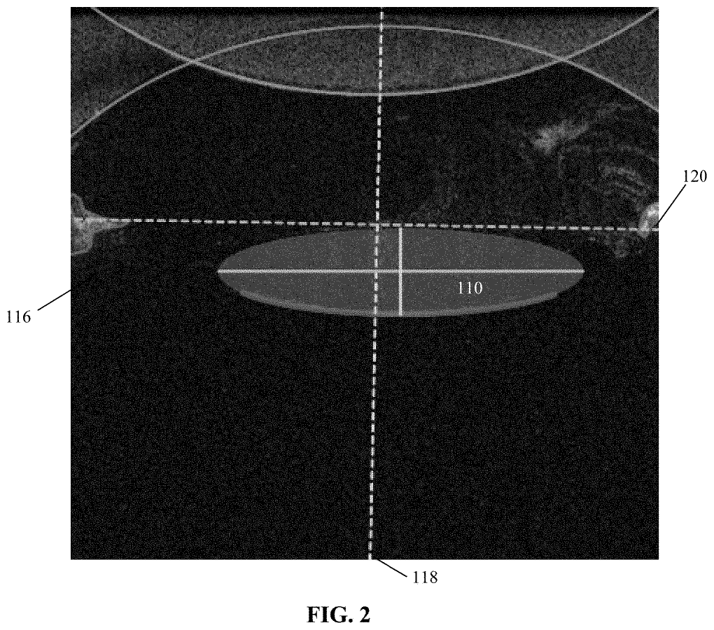

Automated detection of IOL positioning can be performed through optical coherence tomography (OCT) or other imaging modalities to ascertain metrics regarding position and orientation of an IOL inside a capsular bag, as illustrated in FIG. 2. This information is used, in the case of robotic tool control, as feedback to position the IOL 110 and to control and guide the suction-based tool 122 so as to align the IOL axes 108 with the axes 118 and 120 of the eye.

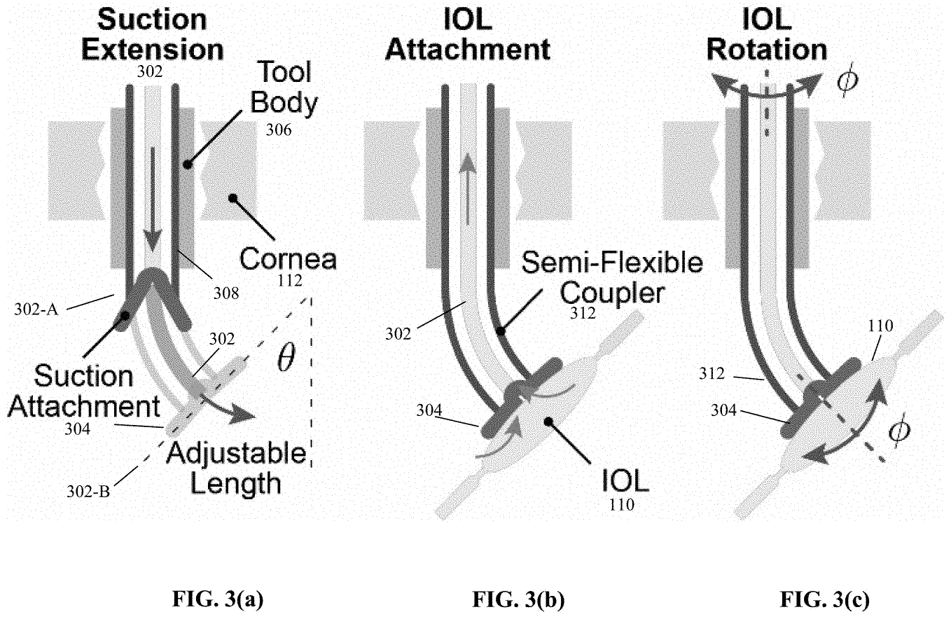

Example aspects of a suction-based tool according to embodiments are further illustrated in FIGS. 3(a) to 3(c). As shown in FIG. 3(a), the tool includes a suction extension 302 and a suction attachment 304 (e.g., a suction pad) at, or connected to, an end of the suction extension as a compliant suction-based end-effector. The suction attachment 304 is sized to accommodate and adhere to optics of an IOL. The extension extends through an internal channel 308 within (e.g. coaxially) and defined by a rigid or flexible tool body 306. The extension 302 extends, retracts, and rotates inside the internal channel, and is used to position the suction attachment 304 (which can be adhered to the IOL) inside a capsular bag. As illustrated in FIG. 3(a), the extension 302 is flexible and includes a preformed bend (e.g. with an angle represented as 0) when the extension is extended away from body 306 by an adjustable amount shown as position 302-B. Meanwhile, as also shown in FIG. 3(a), extension 302 can be straightened when the extension 302 is retracted to position 302-A within the tool body 306. Simultaneously, attachment 304 can fold as shown in FIG. 3(a) so as to be retracted into body 306 along with extension 302. Motion of the extension can be imparted manually, or by a set of actuators (e.g., motors).

As illustrated in FIG. 3(b), a coupler 312 is connected to and surrounds the extension 302 within the internal channel 308 defined by the tool body, and extends, retracts, and rotates along with the extension 302 inside the internal channel 308. As further shown in FIG. 3(b), when coupler 312 and extension 302 are extended into a capsular bag, attachment can be attached directly to optics of IOL 110 via operation of suction (e.g. vacuum) imparted via extension 302. Thereafter, as shown in FIG. 3(c), by manipulation of the tool as imparted via attachment 304 and coupler 312, IOL 110 can be rotated into alignment.

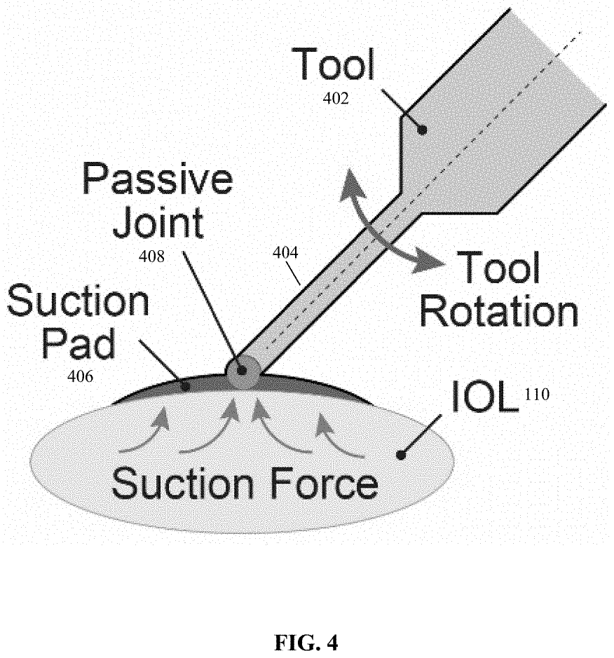

Another example embodiment of a suction-based tool is illustrated in FIG. 4. The tool 402 includes a rigid tool body including a tool tip 404, and a suction attachment 406 (e.g., a suction pad) connected to the tool tip, via a mechanically passive or actuated joint 408 at an end of the tool tip, as a compliant suction-based end-effector. The joint can be, for example, a pivot joint to allow rotation of the suction attachment relative to the tool tip, or a hinge joint to allow motion of the suction attachment in one plane relative to the tool tip. The suction attachment 406 is sized to accommodate and adhere to optics of an IOL 110. Through tool rotation and tool tip positioning, the IOL can be accurately positioned.

For either of the embodiments illustrated in FIG. 3 and FIG. 4, the suction-based tool can be manually controlled by a surgeon, or controlled by a robotic surgical system.

As further embodiments, a suction-based tool can have additional applications, including positioning other types of intraocular implants without the eye. An example includes sub-retinal implants, where a miniaturized wireless photovoltaic sub-retinal implant is injected and should be positioned within a retina. Use of forceps makes this a difficult procedure, but a suction-based tool can be well-suited for this application.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claim(s). In addition, many modifications may be made to adapt a particular situation, material, composition of matter, method, operation or operations, to the objective, spirit and scope of the disclosure. All such modifications are intended to be within the scope of the claim(s) appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

What is claimed is:

1. A suction-based tool for positioning an intraocular implant, comprising:
a tool body defining an internal channel;
a suction extension extending through the internal channel; and
a suction attachment connected to an end of the suction extension.

2. The suction-based tool of claim 1, wherein the suction extension includes a preformed bend that is straightened in a retracted position within the tool body.

3. The suction-based tool of claim 1, further comprising a coupler which is connected to and surrounds the suction extension.

4. The suction-based tool of claim 2, further comprising a coupler which is connected to and surrounds the suction extension.

5. The suction-based tool of claim 1, wherein the suction attachment is sized to accommodate and adhere to optics of an intraocular lens.

6. The suction-based tool of claim 2, wherein the suction attachment is sized to accommodate and adhere to optics of an intraocular lens.

7. The suction-based tool of claim 3, wherein the suction attachment is sized to accommodate and adhere to optics of an intraocular lens.

8. A suction-based tool for positioning an intraocular implant, comprising:
a tool body including a tool tip;
a joint at an end of the tool tip; and
a suction attachment connected to the tool tip via the joint.

9. The suction-based tool of claim 8, wherein the joint is a pivot joint or a hinge joint.

10. The suction-based tool of claim 8, wherein the suction attachment is sized to accommodate and adhere to optics of an intraocular lens.

11. The suction-based tool of claim 9, wherein the suction attachment is sized to accommodate and adhere to optics of an intraocular lens.
